(19) United States
(12) Patent Application Publication
(10) Pub. No.: US 2019/0311377 A1
(43) Pub. Date: Oct. 10, 2019

(54) SOCIAL SECURITY FRAUD BEHAVIORS IDENTIFICATION METHOD, DEVICE, APPARATUS AND COMPUTER-READABLE STORAGE MEDIUM

(71) Applicant: PING AN TECHNOLOGY (SHENZHEN) CO., LTD., Shenzhen, Guangdong (CN)

(72) Inventors: Xiaowen RUAN, Shenzhen City, Guangdong (CN); Liang XU, Shenzhen City, Guangdong (CN); Jing XIAO, Shenzhen City, Guangdong (CN)

(21) Appl. No.: 16/315,089

(22) PCT Filed: Jan. 31, 2018
(86) PCT No.: PCT/CN2018/074806

§ 371 Date: Jan. 3, 2019

(30) Foreign Application Priority Data
Feb. 20, 2017 (CN) 201710091766.9

(51) Int. Cl. G06Q 30/00; G06Q 50/26; G06K 9/62; G16H 15/00; G16H 40/20

(52) U.S. Cl. CPC G06Q 30/0185; G06Q 50/26; G16H 40/20; G16H 15/00; G06K 9/6232

(57) ABSTRACT

Disclosed are a social security fraud behaviors identification method, a social security fraud behaviors identification device and a social security fraud behaviors identification apparatus as well as a computer-readable storage medium. The method includes: establishing a relationship network of doctor-patient and drug diagnosis based on social security medical treatment data, wherein, the relationship network comprises kinds of nodes, the relationship between each node and any other node is different; analyzing group medical treatment behaviors of each node in the relationship network, to extract multiple-dimensional group medical treatment characteristics corresponding to each node; inputting each of the multiple-dimensional group medical treatment characteristics extracted into a preset classification model, to identify fraud rate of each node according to the classification model.

The present application claims the priority to China Patent Application No. 201710091766.9, filed Feb. 20, 2017 with the State Intellectual Property Office and entitled “Social security fraud behaviors Identification Method and Device”, the entirety of which is hereby incorporated herein by reference.

### FIELD

The present disclosure relates to the field of computer application, and more particularly relates to a social security fraud behaviors identification method, a social security fraud behaviors identification device, and a social security fraud behaviors identification apparatus, as well as a computer-readable storage medium.

### BACKGROUND

With the improvement of living standards, people has gradually increased awareness of security assurance about personal safety and security. More and more peoples are willing to purchase social security to reduce the economic burden when accidents occur. Consequently, some social security fraud behaviors also generated, for example, making forgery of medical case, changing medical costs, and so on.

Currently, in most cases, a single rule triggering mechanism is usually used to identify the social security fraud behaviors. However, the single rule triggering mechanism has the defect of using a single evidence chain for identifying the social security fraud behaviors, which is prone to lead to a high rate of misjudgment.

### SUMMARY

It is one main object of the present disclosure to provide a social security fraud behaviors identification method, a social security fraud behaviors identification device, and a social security fraud behaviors identification apparatus as well as a computer-readable storage medium, aiming to solve the technical problem in the prior art that the accuracy for identifying the social security fraud behaviors is low.

In order to realize the above aim, the present disclosure provides a social security fraud behaviors identification method, the method includes:

establishing a relationship network of doctor-patient and drug diagnosis based on social security medical treatment data, wherein, the relationship network comprises kinds of nodes, the relationship between each node and any other node is different;

analyzing group medical treatment behaviors of each node in the relationship network, to extract multiple-dimensional group medical treatment characteristics corresponding to each node;

inputting each of the multiple-dimensional group medical treatment characteristics extracted into a preset classification model, to identify fraud rate of each node according to the classification model.

In addition, in order to realize the above aim, the present disclosure also provides a social security fraud behaviors identification device, the social security fraud behaviors identification device includes:

an establishing module, configured to establish a relationship network of doctor-patient and drug diagnosis based on social security medical treatment data, wherein, the relationship network comprises kinds of nodes, the relationship between each node and any other node is different;

an analyzing and extracting module, configured to analyze group medical treatment behaviors of each node in the relationship network, to extract multiple-dimensional group medical treatment characteristics corresponding to each node;

an input identifying module, configured to input each of the multiple-dimensional group medical treatment characteristics extracted into a preset classification model, to identify fraud rate of each node according to the classification model.

In addition, in order to realize the above aim, the present disclosure also provides a social security fraud behaviors identification apparatus, the social security fraud behaviors identification apparatus includes a processor, a memory storing a social security fraud behaviors identification program; the processor configured to execute the social security fraud behaviors identification program in order to perform above-mentioned steps of the social security fraud behaviors identification method.

In addition, in order to realize the above aim, the present disclosure also provides a computer-readable storage medium that stores a social security fraud behaviors identification program. The social security fraud behaviors identification program is executed by the processor in order to perform above-mentioned steps of the social security fraud behaviors identification method.

In the social security fraud behaviors identification method, the social security fraud behaviors identification device, the social security fraud behaviors identification apparatus and the computer-readable storage medium proposed in the present disclosure, it's first to establish a relationship network of doctor-patient and drug diagnosis based on social security medical treatment data, then to analyze group medical treatment behaviors of each node in the relationship network to extract multiple-dimensional group medical treatment characteristics corresponding to each node, finally to input each of the multiple-dimensional group medical treatment characteristics extracted into a preset classification model, to identify fraud rate of each node according to the classification model. This solution identifies social security fraud behaviors from multiple dimensions and perspectives, compared with the traditional single rule identification, having higher accuracy in identifying social security fraud behaviors.

### BRIEF DESCRIPTION OF THE DRAWINGS

FIG. 1 is an illustrative flowchart of a first embodiment of a social security fraud behaviors identification method in accordance with this disclosure;

FIG. 2 is a detailed illustrative flowchart of the step S10 as illustrated in FIG. 1;

FIG. 3 is a detailed illustrative flowchart of the step S30 as illustrated in FIG. 1;

FIG. 4 is an illustrative flowchart of a second embodiment of a social security fraud behaviors identification method according to this disclosure;

FIG. 5 is a functional module diagram of a first embodiment of a social security fraud behaviors identification device in accordance with this disclosure;

FIG. 6 is a detailed functional module diagram of establishing module 10 as illustrated in FIG. 5;

FIG. 7 is a detailed functional module diagram of input identifying module 30 as illustrated in FIG. 5;

FIG. 8 is a functional module diagram of a second embodiment of a social security fraud behaviors identification device in accordance with this disclosure;

FIG. 9 is a preferred illustration of the relation network in accordance with this disclosure;

FIG. 10 is a schematic diagram of an apparatus in terms of hard operating environment involved in the method embodiments in accordance with this disclosure.

The realizing of the aim, functional characteristics and advantages of the present disclosure are further described in detail with reference to the accompanying drawings and the embodiments.

### DETAILED DESCRIPTION OF THE EMBODIMENTS

It will be appreciated that the specific embodiments described herein are merely illustrative of the present disclosure and are not intended to limit the present disclosure.

It will be appreciated that the existing single rule triggering mechanism refers to the rule triggering mechanism of FWA (Favourite Website Awards) system based on business experience analysis, which performs fraud identification only from single-dimensional data modeling. For example, the FWA system, with limitation on medical treatment expenses, dosage and medical correspondence, identifies medical receipts suspected of fraud by single-dimensional data modeling. However, the above fraud identification has difficulties in identifying fraud cases such as cumulative crimes and group crimes. For example, medication is normal in view of the dosage from the single-dimensional data. But these methods and models are difficult to identify some complex fraud behaviors, such as group payment by social security cards or serial payment by social security cards: a group of people frequently take medicines at different locations for a long time, or a doctor, a department or a hospital has a large number of insured people who frequently pay by their social security cards for a long time.

Based on the problem existing in the prior art, the present disclosure provides a social security fraud behaviors identification method.

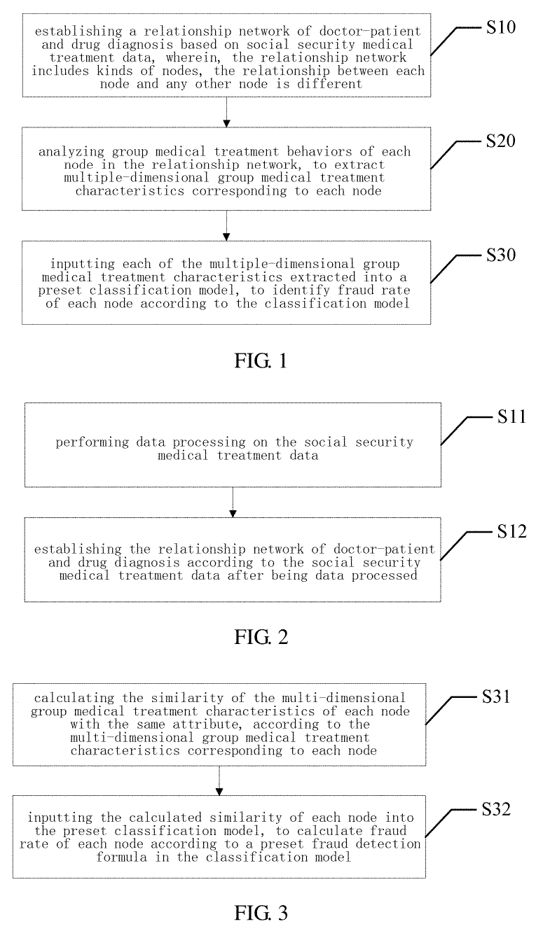

Referring to FIG. 1, FIG. 1 is an illustrative flowchart of a first embodiment of a social security fraud behaviors identification method in accordance with this disclosure.

In the exemplary embodiment, the social security fraud behaviors identification method including:

establishing a relationship network of doctor-patient and drug diagnosis based on social security medical treatment data, wherein, the relationship network comprises kinds of nodes, the relationship between each node and any other node is different; analyzing group medical treatment behaviors of each node in the relationship network, to extract multiple-dimensional group medical treatment characteristics corresponding to each node; inputting each of the multiple-dimensional group medical treatment characteristics extracted into a preset classification model, to identify fraud rate of each node according to the classification model.

The following are the specific steps of a social security fraud behaviors identification method realized step by step according the exemplary embodiment:

S10, establishing a relationship network of doctor-patient and drug diagnosis based on social security medical treatment data, wherein, the relationship network comprises kinds of nodes, the relationship between each node and any other node is different;

In the exemplary embodiment, first social security medical treatment data is obtained from a database, and after the social security medical treatment data is obtained, a relationship network of doctor-patient and drug diagnosis can be established directly based on the social security medical treatment data. The nodes in the relationship network include, but are not limited to: the hospital, the doctor, the patient, the area, the disease, the drug item and so on.

Further, after the social security medical treatment data is obtained, sensitive information processing can be performed on the obtained social security medical treatment data. The sensitive information processing refers to data deformation of sensitive information in the data with a sensitive information processing rule, for the protection of sensitive privacy data. Subsequently, the relationship network of doctor-patient and drug diagnosis can be established based on the social security medical treatment data after being sensitive information processed. Preferably, all the social security medical treatment data below is the social security medical treatment data after being sensitive information processed, thus they are not to be detailed herein.

Specifically, referring to FIG. 2, the step S10 includes:

S11, performing data processing on the social security medical treatment data;

S12, establishing the relationship network of doctor-patient and drug diagnosis according to the social security medical treatment data after being data processed.

In the exemplary embodiment, after obtaining the social security medical treatment data, it's first to perform data processing on the social security medical treatment data. The data processing may include de-noising and de-interference of the data, so as to facilitate the relationship network established later more accurate. After performing data processing on the social security medical treatment data, the relationship network of doctor-patient and drug diagnosis is established according to the social security medical treatment data after being data processed.

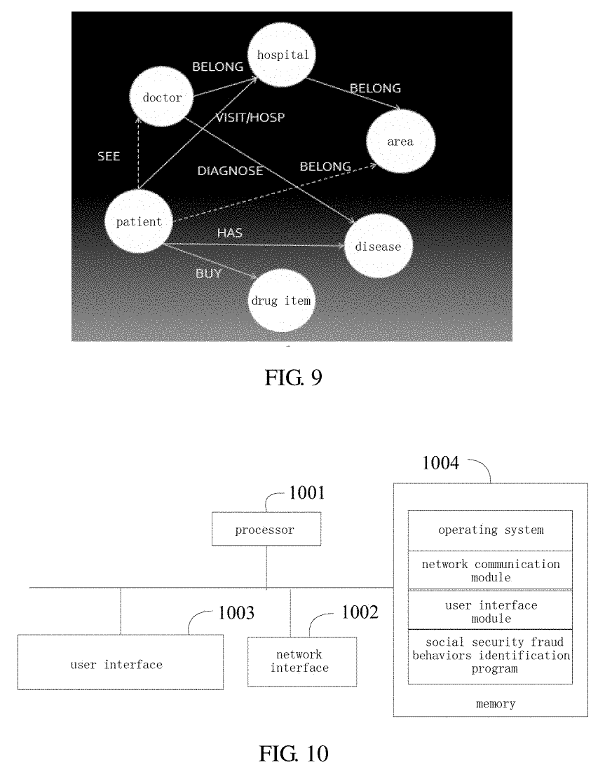

In the exemplary embodiment, the relationship network established based on the social security medical treatment data may be referred to FIG. 9. As illustrated in FIG. 9, the relationship network includes multiple nodes which are: the hospital, the doctor, the patient, the area, the disease and the drug item and so on. It can be seen in FIG. 9, in the relationship network, the relationship between one node and any other node is different. For example, the relationship between the doctor and the hospital is that the doctor belongs to the hospital; the relationship between the doctor and the disease is that the doctor diagnoses the disease; the relationship between the patient and the drug item is that the patient buys the drug item; the relationship between the patient and the disease is that the patient has the disease, and so on. With the relationship network, medical treatment behaviors of the patient can be monitored in all aspects.

It will be understood that, the relationship network illustrated in FIG. 9 is merely a preferred illustration of this embodiment, and the relationship network shown in FIG. 9 is only a small part of the relationship network of this embodiment. As can be seen from the relationship network illustrated in FIG. 9, each node is a different type of node, so each node is a node with a different attribute. However, the relationship network of this embodiment may actually include multiple nodes having the same attribute, such as multiple nodes of the doctor, or multiple nodes of the patient, and the relationship between a node and any other node having the same attribute is also different. Therefore, the nodes in this embodiment are not limited to the above-mentioned content. In the case of the social security medical treatment data changing, different relationship networks and nodes will also be obtained, thus they are not to be detailed herein.

S20, analyzing group medical treatment behaviors of each node in the relationship network, to extract multiple-dimensional group medical treatment characteristics corresponding to each node;

In this embodiment, after establishing the relationship network of doctor-patient and drug diagnosis based on the social security medical treatment data, group medical treatment behaviors of each node in the relationship network are analyzed. In this embodiment, the analysis of group medical treatment behaviors of each node in the relationship network, continuing to take FIG. 9 as an example, is to analyze medical treatment behaviors presented in the relationship network, which is equivalent to analyzing medical behaviors of the patient, analyzing treatment behaviors of the doctor or analyzing treatment means of the disease, and so on. The relationship between one node and any other node is different, and each node is no longer affected by a single dimension, but affected by a comprehensive influence of the other nodes in the relationship network, therefore, with analysis of the group medical treatment behaviors of each node, multiple-dimensional group medical treatment characteristics of each node can be eventually obtained. The medical treatment characteristics are characteristics extracted from the medical behaviors. Taking the patient node in FIG. 9 for example, the group medical treatment characteristics of the patient node include: areas where the patient belongs to, hospitals where the patient sees a doctor, the number and specific time of the patient purchasing drug items, diseases which the patient has, doctors who diagnose the patient, and so on. The group medical treatment behaviors analysis of the patient is equivalent to a comprehensive analysis of areas where the patient belongs to, the number and specific time of the patient buying drug items, and diseases which the patient has and so on. If it is found that a patient has bought a large number of drugs in different hospitals many times, and the types of the drugs are different, the group medical treatment characteristics can be determined as: the amount of drugs bought by the user is large, there is many types of drugs, and so on.

S30, inputting each of the multiple-dimensional group medical treatment characteristics extracted into a preset classification model, to identify fraud rate of each node according to the classification model.

After extracting the multiple-dimensional group medical treatment characteristics corresponding to each node, each of the group medical treatment characteristics extracted is input into a preset classification model, in order to identify fraud rate of each node according to the classification model. Specifically, referring to FIG. 3, the step S30 includes:

S31, calculating the similarity of the multi-dimensional group medical treatment characteristics of each node having the same attribute, according to the multi-dimensional group medical treatment characteristics corresponding to each node;

S32, inputting the calculated similarity of each node into the preset classification model, to calculate fraud rate of each node according to a preset fraud detection formula in the classification model.

That is to say, after extracting the multi-dimensional group medical treatment characteristics corresponding to each node, the similarity of the multi-dimensional group medical treatment characteristics of each node having the same attribute is calculated. The nodes having the same attribute are such as: a doctor node and another doctor node, or a patient node and another patient node.

In the exemplary embodiment, the calculating the similarity of the multi-dimensional group medical treatment characteristics of each node having the same attribute preferably takes the following algorithms:

1) Jaccard Similarity (representing the generalized similarity):

Jaccard(A, B)=|A intersect B|/|A union B|

wherein, Intersect represents intersection, Union represents union, A and B represent nodes having the same attribute, for example, A and B both represent the doctor node in FIG. 9, or both represent the patient node.

2) Euclidean similarity (similarity of Euclidean distance):

Euclidean(A, B)=1−euclidean_distance(A, B)

wherein, A and B represent nodes having the same attribute.

The two algorithms listed above for calculating the similarity of the multi-dimensional group medical treatment characteristics of each node having the same attribute are merely illustrative. Other algorithms proposed by those skilled in the art by taking use of the technical idea of the present disclosure and according to their specific requirements are within the protection scope of the present disclosure. Thus, they are not to be detailed herein.

By the above similarity calculation formulas, the similarity of the multi-dimensional group medical treatment characteristics of any two nodes having the same attribute can be determined.

After determining the similarity of the multiple-dimensional group medical treatment characteristics of each node of the nodes having the same attribute, the calculated similarity of each node is input into a preset classification model, in order to calculate fraud rate of each node in accordance with a preset fraud detection formula in the classification model. Wherein, the fraud detection formula preferably includes: KNN (k-Nearest Neighbor algorithm, K taking the value 5) algorithm formula; binary Kmeans algorithm formula; Shewhart methods algorithm formula, and so on. For these algorithm formulas are existing formulas, the calculating processes are not to be detailed herein.

Further, in order to improve accuracy of the classification model calculating fraud rate of nodes, in the exemplary embodiment, after the step S32, the social security fraud behaviors identification method further includes:

step A, verifying the fraud rate of each node, and adding the verification conclusion to the fraud rate of each node;

step B, re-inputting the fraud rate with the verification conclusion into the classification model for training the classification model.

That is to say, after calculating fraud rate of each node according to the preset fraud detection formula in the classification model, the fraud rate can also be verified. In this embodiment, the way of verification is preferably an offline approval verification. After verifying the fraud rate of each node, the verification conclusion is added into the fraud rate of each node and the fraud rate with the verification conclusion is re-input into the classification model for training the classification model, which makes the classification model more accurate in identifying the fraud rate of nodes subsequently.

The social security fraud behaviors identification based on the relationship network according to this embodiment is, from group dimensions, to establish a relationship network of medical treatment for group's medical treatment behaviors, and to design an algorithm model for identifying fraud behaviors from the group dimensions, so as to obtain fraud rate of nodes, which realizes the identification of social security behaviors of the group dimensions. It will be understood that, with the analysis of user's social security medical treatment data, if the fraud rate of multiple nodes are detected to be high, only a few nodes with low fraud rate, the user may be considered to have social security fraud behaviors. Compared with the single rule triggering mechanism, the social security fraud behaviors identification, which determining whether a user has social security fraud behaviors by the group medical treatment behaviors, is more accurate.

In the social security fraud behaviors identification method proposed in this embodiment, it's first to establish a relationship network of doctor-patient and drug diagnosis based on social security medical treatment data, then to analyze group medical treatment behaviors of each node in the relationship network, in order to extract multiple-dimensional group medical treatment characteristics, finally to input each of the multiple-dimensional group medical treatment characteristics extracted into a preset classification model, in order to identify fraud rate of each node according to the classification model. This solution identifies social security fraud behaviors from multiple dimensions and perspectives, compared with the traditional single rule identification, having higher accuracy in identifying social security fraud behaviors.

Further, in order to improve accuracy of the social security fraud behaviors identification, based on the first embodiment, a second embodiment of the social security fraud behaviors identification method is provided.

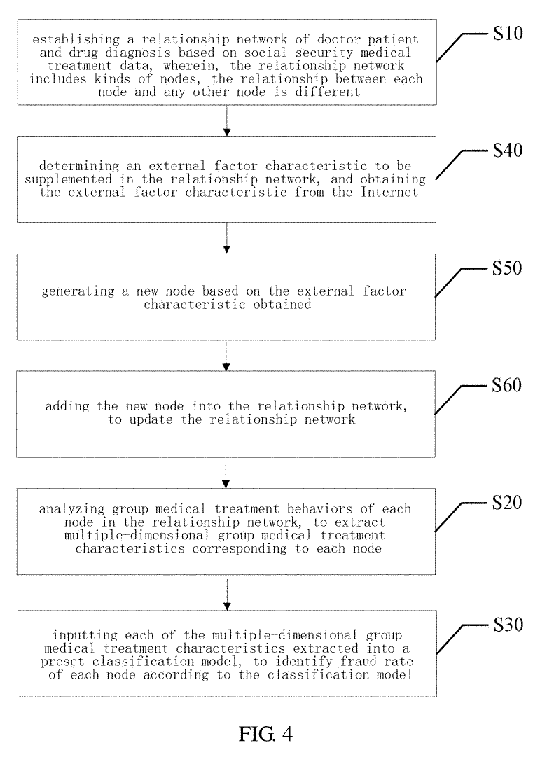

In the exemplary embodiment, referring to FIG. 4, before the step S20, the social security fraud behaviors identification method further includes:

S40, determining an external factor characteristic to be supplemented in the relationship network, and obtaining the external factor characteristic from the Internet;

S50, generating a new node based on the external factor characteristic obtained;

S60, adding the new node into the relationship network, to update the relationship network.

In the exemplary embodiment, an external factor characteristic to be supplemented is determined in the relationship network, and the external factor characteristic is obtained from the Internet. The external factor characteristic refers to external information related to the node. For example, if the node is the hospital, the external factor characteristic is the information related to the hospital, such as address information of the hospital and so on. After obtaining the external factor characteristic, it's first to generate a new node based on the external factor characteristic obtained, finally to add the new node into the relationship network so as to update the relationship network. These make the nodes more detailed in the subsequent relationship network and the identification of the fraud rate of each subsequent node more accurate.

In should be noted that, although each algorithm involved is an existing algorithm, the entire operations adopted in the whole process of social security fraud behaviors identification are different from that in the existing social security fraud behaviors identification. The present disclosure overcomes the problem that the accuracy for identifying the social security fraud behaviors is low.

It will be clarified that, those skilled in the art can understand that all or part of the steps in the above embodiments may be implemented by hardware, or by a program instructing related hardware. The program may be stored in a computer-readable storage medium, and the above-mentioned storage medium may be a read only memory, a magnetic disk or an optical disk, and so on.

The present disclosure further provides a social security fraud behaviors identification device.

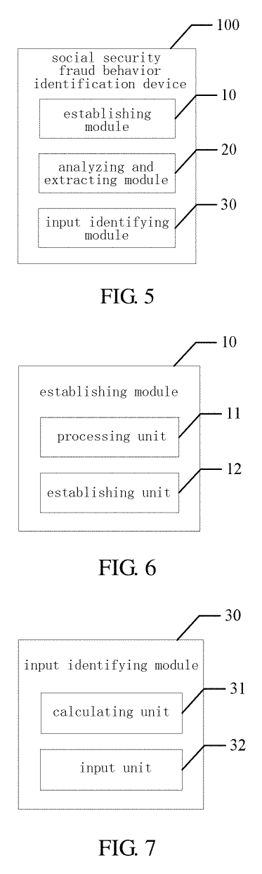

Referring to FIG. 5, FIG. 5 is a functional module diagram of a first embodiment of a social security fraud behaviors identification device in accordance with this disclosure.

It should be stressed that, for those skilled in the art, the schematic diagram shown in FIG. 5 is merely an exemplary diagram of a preferable embodiment. Those skilled in the art may easily supplement new functional modules by centering on the functional module of the social security fraud behaviors identification device 100 shown in FIG. 5. The name of each functional module is custom name, which is merely used to aid understanding of each program functional module of the social security fraud behaviors identification device 100, and is not used to limit the technical solution of the present disclosure. The core of the technical solution of the present disclosure is the function to be achieved by each functional module with the custom name.

In the exemplary embodiment, the social security fraud behaviors identification device 100 includes:

an establishing module 10, configured to establish a relationship network of doctor-patient and drug diagnosis based on social security medical treatment data, wherein, the relationship network comprises kinds of nodes, the relationship between each node and any other node is different;

an analyzing and extracting module 20, configured to analyze group medical treatment behaviors of each node in the relationship network, to extract multiple-dimensional group medical treatment characteristics corresponding to each node;

an input identifying module 30, configured to input each of the multiple-dimensional group medical treatment characteristics extracted into a preset classification model, to identify fraud rate of each node according to the classification model.

In the exemplary embodiment, first social security medical treatment data is obtained from a database, and after the social security medical treatment data is obtained, the establishing module 100 can directly establishes a relationship network of doctor-patient and drug diagnosis based on the social security medical treatment data. The nodes in the relationship network include, but are not limited to: the hospital, the doctor, the patient, the area, the disease, the drug item and so on.

Further, after the social security medical treatment data is obtained, sensitive information processing can be performed on the obtained social security medical treatment data. The sensitive information processing refers to data deformation of sensitive information in the data with a sensitive information processing rule, for the protection of sensitive privacy data. Subsequently, the establishing module 10 can establish the relationship network of doctor-patient and drug diagnosis based on the social security medical treatment data after being sensitive information processed. Preferably, all the social security medical treatment data below is the social security medical treatment data after being sensitive information processed, thus they are not to be detailed herein.

Specifically, referring to FIG. 6, the establishing module 10 includes:

a processing unit 11, configured to perform data processing on the social security medical treatment data;

a establishing unit 12, configured to establish the relationship network of doctor-patient and drug diagnosis according to the social security medical treatment data after being data processed.

In the exemplary embodiment, after obtaining the social security medical treatment data, the processing unit 11 is configured to first perform data processing on the social security medical treatment data. The data processing may include de-noising and de-interference of the data, so as to facilitate the relationship network established later more accurate. After performing data processing on the social security medical treatment data, the establishing unit 12 is configured to establish the relationship network of doctor-patient and drug diagnosis according to the social security medical treatment data after being data processed.

In the exemplary embodiment, the relationship network established based on the social security medical treatment data may be referred to FIG. 9. As illustrated in FIG. 9, the relationship network includes multiple nodes which are: the hospital, the doctor, the patient, the area, the disease and the drug item and so on. It can be seen in FIG. 9, in the relationship network, the relationship between one node and any other node is different. For example, the relationship between the doctor and the hospital is that the doctor belongs to the hospital; the relationship between the doctor and the disease is that the doctor diagnoses the disease; the relationship between the patient and the drug item is that the patient buys the drug item; the relationship between the patient and the disease is that the patient has the disease, and so on. With the relationship network, medical treatment behaviors of the patient can be monitored in all aspects.

It will be understood that, the relationship network illustrated in FIG. 9 is merely a preferred illustration of this embodiment, and the relationship network shown in FIG. 9 is only a small part of the relationship network of this embodiment. As can be seen from the relationship network illustrated in FIG. 9, each node is a different type of node, so each node is a node with a different attribute. However, the relationship network of this embodiment may actually include multiple nodes having the same attribute, such as multiple nodes of the doctor, or multiple nodes of the patient, and the relationship between a node and any other node having the same attribute is also different. Therefore, the nodes in this embodiment are not limited to the above-mentioned content. In the case of the social security medical treatment data changing, different relationship networks and nodes will also be obtained, thus they are not to be detailed herein.

In this embodiment, after the establishing module 10 establishes a relationship network of doctor-patient and drug diagnosis based on the social security medical treatment data, the analyzing and extracting module 20 analyzes group medical treatment behaviors of each node in the relationship network. In this embodiment, the analyzing and extracting module 20 analyzing group medical behaviors of each node in the relationship network, continuing to take FIG. 9 as an example, is to analyze medical treatment behaviors presented in the relationship network, which is equivalent to analyzing medical behaviors of the patient, analyzing treatment behaviors of the doctor or analyzing treatment means of the disease, and so on. The relationship between one node and any other node is different, and each node is no longer affected by a single dimension, but affected by a comprehensive influence of the other nodes in the relationship network, therefore, with analysis of the group medical treatment behaviors of each node, multiple-dimensional group medical treatment characteristics of each node can be eventually obtained. The medical treatment characteristics are characteristics extracted from the medical behaviors.

Taking the patient node in FIG. 9 for example, the group medical treatment characteristics of the patient node include: areas where the patient belongs to, hospitals where the patient sees a doctor, the number and specific time of the patient purchasing drug items, diseases which the patient has, doctors who diagnose the patient, and so on. The group medical treatment behaviors analysis of the patient is equivalent to a comprehensive analysis of areas where the patient belongs to, the number and specific time of the patient buying drug items, and diseases which the patient has and so on. If it is found that a patient has bought a large number of drugs in different hospitals many times, and the types of the drugs are different, the group medical treatment characteristics can be determined as: the amount of drugs bought by the user is large, there is many types of drugs, and so on.

After the analyzing and extracting module 20 extracting the multiple-dimensional group medical treatment characteristics corresponding to each node, the input identifying module 30 inputs each of the multiple-dimensional group medical treatment characteristics extracted into a preset classification model, to identify fraud rate of each node according to the classification model. Specifically, referring to FIG. 7, the input identifying module 30 includes:

a calculating unit 31, configured to calculate the similarity of the multi-dimensional group medical treatment characteristics of each node having the same attribute, according to the multi-dimensional group medical treatment characteristics corresponding to each node;

an input unit 32, configured to input the calculated similarity of each node into the preset classification model, to calculate fraud rate of each node according to a preset fraud detection formula in the classification model; and

the calculating unit 31 also being configured to calculate fraud rate of each node according to a preset fraud detection formula in the classification model.

That is to say, after extracting the multi-dimensional group medical treatment characteristics corresponding to each node, the calculating unit 31 calculates the similarity of the multi-dimensional group medical treatment characteristics of each node having the same attribute. The nodes having the same attribute are such as: a doctor node and a doctor node, or a patient node and a patient node.

In the exemplary embodiment, the calculating unit 31 calculates the similarity of the multi-dimensional group medical treatment characteristics of each node having the same attribute preferably takes the following algorithms:

1) Jaccard Similarity (representing the generalized similarity):

Jaccard(A, B)=|A intersect B|/|A union B|

wherein, Intersect represents intersection, Union represents union, A and B represent nodes having the same attribute, for example, A and B both represent the doctor node in FIG. 9, or both represent the patient node.

2) Euclidean similarity (similarity of Euclidean distance):

Euclidean(A, B)=1−euclidean_distance(A, B)

wherein, A and B represent nodes having the same attribute.

The two algorithms listed above for calculating the similarity of the multi-dimensional group medical treatment characteristics of each node with the same attribute are merely illustrative. Other algorithms proposed by those skilled in the art by taking use of the technical idea of the present disclosure and according to their specific requirements are within the protection scope of the present disclosure. Thus, they are not to be detailed herein.

By the above similarity calculation formulas, the similarity of the multi-dimensional group medical treatment characteristics of any two nodes having the same attribute can be determined.

That is to say, after extracting the multi-dimensional group medical treatment characteristics corresponding to each node, the similarity of the multi-dimensional group medical treatment characteristics of each node having the same attribute is calculated. The nodes having the same attribute are such as: a doctor node and another doctor node, or a patient node and another patient node, namely, the nodes with the same type represent the nodes having the same attribute.

In the exemplary embodiment, the calculating the similarity of the multi-dimensional group medical treatment characteristics of each node having the same attribute preferably takes the following algorithms:

1) Jaccard Similarity (representing the generalized similarity):

Jaccard(A, B)=|A intersect B|/|A union B|

wherein, Intersect represents intersection, Union represents union, A and B represent nodes having the same attribute, for example, A and B both represent nodes of the doctor in FIG. 9, or both represent nodes of the patient.

2) Euclidean similarity (similarity of Euclidean distance):

Euclidean(A, B)=1−euclidean_distance(A, B)

wherein, A and B represent nodes having the same attribute.

After determining the similarity of the multiple-dimensional group medical treatment characteristics of each node having the same attribute, the inputting unit 32 inputs the calculated similarity of each node into a preset classification model, to calculate fraud rate of each node in accordance with a preset fraud detection formula in the classification model. Wherein, the fraud detection formula preferably includes: KNN (k-Nearest Neighbor algorithm, K taking the value 5) algorithm formula; binary Kmeans algorithm algorithm formula; Shewhart methods algorithm algorithm formula, and so on. For these algorithm formulas are existing formulas, the calculating processes are not to be detailed herein.

Further, in order to improve accuracy of the classification model calculating fraud rate of nodes, in the exemplary embodiment, the social security fraud behaviors identification device 100 further includes:

a verifying module, configured to verify the fraud rate of each node, and adding the verification conclusion to the fraud rate of each node;

a training module, configured to re-input the fraud rate with the verification conclusion into the classification model for training the classification model.

That is to say, after calculating fraud rate of each node according to the preset fraud detection formula in the classification model, the verifying module can also verify the fraud rate. In this embodiment, the way of verification is preferably an offline approval verification. After verifying the fraud rate of each node, the verification conclusion is added into the fraud rate of each node and the fraud rate with the verification conclusion is re-input into the classification model for training the classification model, which makes the classification model more accurate in identifying the fraud rate of nodes subsequently.

The social security fraud behaviors identification based on the relationship network according to this embodiment is, from group dimensions, to establish a relationship network of medical treatment for group's medical treatment behaviors, and to design an algorithm model for identifying fraud behaviors from the group dimensions, so as to obtain fraud rate of nodes, which realizes the identification of social security behaviors of the group dimensions. It will be understood that, with the analysis of user's social security medical treatment data, if the fraud rate of multiple nodes are detected to be high, only a few nodes with low fraud rate, the user may be considered to have social security fraud behaviors. Compared with the single rule triggering mechanism, the social security fraud behaviors identification, which determining whether a user has social security fraud behaviors by the group medical treatment behaviors, is more accurate.

The social security fraud behaviors identification device 100 proposed in the exemplary embodiment, first establishes a relationship network of doctor-patient and drug diagnosis based on the social security medical treatment data, then analyzes group medical treatment behaviors of each node in the relationship network, in order to extract multiple-dimensional group medical treatment characteristics, finally inputs each of the multiple-dimensional group medical treatment characteristics extracted into a preset classification model, in order to identify fraud rate of each node according to the classification model. This solution identifies social security fraud behaviors from multiple dimensions and perspectives, compared with the traditional single rule identification, having higher accuracy in identifying social security fraud behaviors.

Further, in order to improve accuracy of the social security fraud behaviors identification, based on the first embodiment, a second embodiment of the social security fraud behaviors identification device 100 according to the present disclosure is provided.

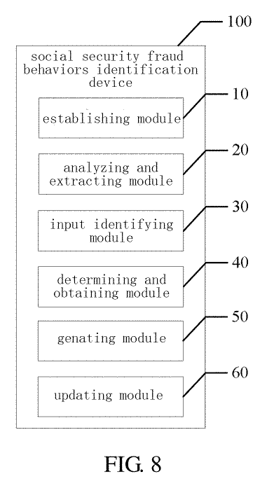

In the exemplary embodiment, referring to FIG. 8, the social security fraud behaviors identification device 100 further includes:

a determining and obtaining module 40, configured to determine an external factor characteristic to be supplemented in the relationship network, and obtaining the external factor characteristic from the Internet;

a generating module 50, configured to generate a new node based on the external factor characteristic obtained;

a updating module 60, configured to add the new node into the relationship network, to update the relationship network.

In the exemplary embodiment, first the determining and obtaining module 40 determines an external factor characteristic to be supplemented in the relationship network, and obtains the external factor characteristic from the Internet. The external factor characteristic refers to external information related to a node. For example, if the node is the hospital, the external factor characteristic is the information related to the hospital, such as address information of the hospital and so on. After obtaining the external factor characteristic, the generating module 50 generates a new node based on the external factor characteristic obtained, and the updating module 60 adds the new node into the relationship network so as to update the relationship network. These make the nodes more detailed in the subsequent relationship network and the identification of the fraud rate of each subsequent node more accurate.

In should be noted that, although each algorithm involved is an existing algorithm, the entire operations adopted in the whole process of social security fraud behaviors identification are different from that in the existing social security fraud behaviors identification. The present disclosure overcomes the problem that the accuracy for identifying the social security fraud behaviors is low.

It should be clarified that, in terms of hardware implementation, the above establishing module 10, analyzing and extracting module 20 and input identifying module 30 and the like may be embedded in or independent of the social security fraud behaviors identification device, or be stored in the social security fraud behaviors identification device in the form of software so as for the processor to execute operations corresponding to each module above. The processor may be a central processing unit (CPU), a microprocessor, a microcontroller and so on.

Referring to FIG. 10, FIG. 10 is a schematic diagram of an apparatus in terms of hard operating environment involved in the method embodiments in accordance with this disclosure.

The social security fraud behaviors identification apparatus according to the embodiments of the present disclosure may be a PC, or may be a terminal device such as a smart phone, a tablet computer, or a portable computer and so on.

As illustrated in FIG. 10, the social security fraud behaviors identification apparatus may include: a processor 1001, such as CPU, a network interface 1002, a user interface 1003, a memory 1004. Communications between these components can be implemented by a communication bus. The network interface 1002 may optionally include a standard wired interface (configured to connect a wired network), and a wireless interface (such as a WI-FI interface, a Bluetooth interface, an infrared interface and the like, configured to connect to a wireless network). The user interface 1003 may include a display, an input unit such as a keyboard, and an optional user interface 1003 may also include a standard wired interface (such as configured to connect to a wired keyboard, a wired mouse and so on) and wireless interface (such as configured to connect to a wireless keyboard, a wireless mouse). The memory 1004 may be a high-speed RAM memory, or a non-volatile memory such as a disk memory. The memory 1004 optionally may also be a storage device that is separate from the processor 1001 described above.

Optionally, the social security fraud behaviors identification apparatus may also include a camera, a RF (Radio Frequency) circuitry, a sensor, an audio circuitry, a WiFi module, and the like.

Those skilled in the art may understand that the structure of the social security fraud behaviors identification apparatus illustrated in FIG. 10 does not constitute a limitation on the apparatus. Thus, the apparatus may include more or less components than those illustrated, or some components to be combined, or different arrangements of components

As illustrated in FIG. 10, the memory 1004 as a computer storage medium may include an operating system, a network communication module, a user interface module and a social security fraud behaviors identification program. The operating system is a program that manages and controls hardware and software resources of the social security fraud behaviors identification device, as well as supports operations of the network communication module, the user interface module, the social security fraud behaviors identification program and other programs or software. The network communication module is configured to manage and control the network interface 1002. The user interface module is configured to manage and control the user interface 1003.

In the social security fraud behaviors identification device illustrated in FIG. 10, the processor 1001 may be configured to execute the social security fraud behaviors identification program stored in the memory 1004, in order to perform each step of the social security fraud behaviors identification method described above.

The present disclosure provides a computer-readable storage medium. The computer-readable storage medium stores a social security fraud behaviors identification program which is executed by the processor to perform each step of the social security fraud behaviors identification method described above.

It's to be clarified that the term “include”, “comprise” or any other variants thereof is intended to encompass a non-exclusive inclusion, such that a process, method, device, or system including a series of elements not only includes those elements, but also includes other elements that are not explicitly listed, or elements that are inherent to such a process, method, device, or system. In the absence of more restrictions, an element defined by the phrase “including one . . . ” does not exclude the existence of additional identical elements in the process, method, device, or system that includes the element.

The numbers of the embodiments according to the present disclosure are merely for description, and do not represent for the advantages and disadvantages of the embodiments.

The foregoing description merely portrays some illustrative embodiments in accordance with the disclosure and therefore is not intended to limit the patentable scope of the disclosure. Any equivalent structure or flow transformations that are made taking advantage of the specification and accompanying drawings of the disclosure and any direct or indirect applications thereof in other related technical fields shall all fall in the scope of protection of the disclosure.

1. A social security fraud behaviors identification method, comprising:
establishing a relationship network of doctor-patient and drug diagnosis based on social security medical treatment data, wherein, the relationship network comprises kinds of nodes, the relationship between each node and any other node is different;
analyzing group medical treatment behaviors of each node in the relationship network, to extract multiple-dimensional group medical treatment characteristics corresponding to each node;
inputting each of the multiple-dimensional group medical treatment characteristics extracted into a preset classification model, to identify fraud rate of each node according to the classification model.

2. The method of claim 1, wherein the step of establishing a relationship network of doctor-patient and drug diagnosis based on social security medical treatment data comprises:
performing data processing on the social security medical treatment data;
establishing the relationship network of doctor-patient and drug diagnosis according to the social security medical treatment data after being data processed.

3. The method of claim 1, wherein the step of inputting each of the multiple-dimensional group medical treatment characteristics extracted into a preset classification model, to identify fraud rate of each node according to the classification model comprises:
calculating the similarity of the multi-dimensional group medical treatment characteristics of each node having the same attribute, according to the multi-dimensional group medical treatment characteristics corresponding to each node;
inputting the calculated similarity of each node into the preset classification model, to calculate fraud rate of each node according to a preset fraud detection formula in the classification model.

4. The method of claim 3, wherein after the step of calculating fraud rate of each node according to a preset fraud detection formula in the classification model, the social security fraud behaviors identification method further comprises:
verifying the fraud rate of each node to add the verification conclusion to the fraud rate of each node;
re-inputting the fraud rate with the verification conclusion into the classification model for training the classification model.

5. The method of claim 1, wherein before the step of analyzing group medical treatment behaviors of each node in the relationship network, to extract multiple-dimensional group medical treatment characteristics corresponding to each node, the social security fraud behaviors identification method further comprises:
determining an external factor characteristic to be supplemented in the relationship network, and obtaining the external factor characteristic from the Internet;
generating a new node based on the external factor characteristics obtained; and
adding the new node into the relationship network, to update the relationship network.

6-10. (canceled)

11. A social security fraud behaviors identification apparatus, comprising a processor, and a memory storing a social security fraud behaviors identification program; the processor is configured to execute the social security fraud behaviors identification program to perform the following steps:
establishing a relationship network of doctor-patient and drug diagnosis based on social security medical treatment data, wherein, the relationship network comprises kinds of nodes, the relationship between each node and any other node is different;
analyzing group medical treatment behaviors of each node in the relationship network, to extract multiple-dimensional group medical treatment characteristics corresponding to each node;
inputting each of the multiple-dimensional group medical treatment characteristics extracted into a preset classification model, to identify fraud rate of each node according to the classification model.

12. The apparatus of claim 11, wherein the processor is also configured to execute the social security fraud behaviors identification program to perform the step of establishing a relationship network of doctor-patient and drug diagnosis based on social security medical treatment data:
performing data processing on the social security medical treatment data;
establishing the relationship network of doctor-patient and drug diagnosis according to the social security medical treatment data after being data processed.

13. The apparatus of claim 11, wherein the processor is also configured to execute the social security fraud behaviors identification program to perform the step of inputting each of the multiple-dimensional group medical treatment characteristics extracted into a preset classification model, to identify fraud rate of each node according to the classification model:
calculating the similarity of the multi-dimensional group medical treatment characteristics of each node having the same attribute, according to the multi-dimensional group medical treatment characteristics corresponding to each node;
inputting the calculated similarity of each node into the preset classification model, to calculate fraud rate of each node according to a preset fraud detection formula in the classification model.

14. The apparatus of claim 13, wherein after the step of calculating fraud rate of each node according to a preset fraud detection formula in the classification model, the processor is also configured to execute the social security fraud behaviors identification program to perform the following steps:
verifying the fraud rate of each node to add the verification conclusion to the fraud rate of each node;
re-inputting the fraud rate with the verification conclusion into the classification model for training the classification model.

15. The apparatus of claim 11, wherein before the step of analyzing group medical treatment behaviors of each node in the relationship network, to extract multiple-dimensional group medical treatment characteristics corresponding to each node, the processor is also configured to execute the social security fraud behaviors identification program to perform the following steps:
determining an external factor characteristic to be supplemented in the relationship network, and obtaining the external factor characteristic from the Internet;
generating a new node based on the external factor characteristics obtained; and
adding the new node into the relationship network, to update the relationship network.

16. A computer-readable storage medium, the computer-readable storage medium storing a social security fraud behaviors identification program, the social security fraud behaviors identification program when being executed by a processor performing the following steps:
establishing a relationship network of doctor-patient and drug diagnosis based on social security medical treatment data, wherein, the relationship network comprises kinds of nodes, the relationship between each node and any other node is different;
analyzing group medical treatment behaviors of each node in the relationship network, to extract multiple-dimensional group medical treatment characteristics corresponding to each node;
inputting each of the multiple-dimensional group medical treatment characteristics extracted into a preset classification model, to identify fraud rate of each node according to the classification model.

17. The computer-readable storage medium of claim 16, wherein the social security fraud behaviors identification program when being executed by a processor also performing the step of establishing a relationship network of doctor-patient and drug diagnosis based on social security medical treatment data:
performing data processing on the social security medical treatment data;
establishing the relationship network of doctor-patient and drug diagnosis according to the social security medical treatment data after being data processed.

18. The computer-readable storage medium of claim 16, wherein the social security fraud behaviors identification program when being executed by a processor also performing the step of inputting each of the multiple-dimensional group medical treatment characteristics extracted into a preset classification model, to identify fraud rate of each node according to the classification mode:
calculating the similarity of the multi-dimensional group medical treatment characteristics of each node having the same attribute, according to the multi-dimensional group medical treatment characteristics corresponding to each node;
inputting the calculated similarity of each node into the preset classification model, to calculate fraud rate of each node according to a preset fraud detection formula in the classification model.

19. The computer-readable storage medium of claim 18, wherein after the step of calculating fraud rate of each node according to a preset fraud detection formula in the classification model, the social security fraud behaviors identification program when being executed by a processor also performing the following steps:
verifying the fraud rate of each node to add the verification conclusion to the fraud rate of each node;
re-inputting the fraud rate with the verification conclusion into the classification model for training the classification model.

20. The computer-readable storage medium of claim 16, wherein before the step of analyzing group medical treatment behaviors of each node in the relationship network, to extract multiple-dimensional group medical treatment characteristics corresponding to each node, the social security fraud behaviors identification program when being executed by a processor also performing the following steps:
determining an external factor characteristic to be supplemented in the relationship network, and obtaining the external factor characteristic from the Internet;
generating a new node based on the external factor characteristics obtained; and
adding the new node into the relationship network, to update the relationship network.
